(19) United States
(12) Patent Application Publication
(10) Pub. No.: US 2026/0166337 A1
(43) Pub. Date: Jun. 18, 2026

(54) AUTOMATED BRACHYTHERAPY TREATMENT PLANNING USING KNOWLEDGE-BASED DOSE ESTIMATIONS

(71) Applicant: The Regents of the University of California, Oakland, CA (US)

(72) Inventors: Sandra Meyers, San Diego, CA (US); Kevin Lawrence Moore, San Diego, CA (US)

(21) Appl. No.: 19/125,697

(22) PCT Filed: Nov. 17, 2023
(86) PCT No.: PCT/US2023/080346

§ 371 Date: Apr. 29, 2025

Related U.S. Application Data
(60) Provisional application No. 63/384,302, filed on Nov. 18, 2022.

(51) Int. Cl. A61N 5/10

(52) U.S. Cl. CPC A61N 5/1031; A61N 5/1001

(57) ABSTRACT

At least one anatomical mask and an applicator mask are provided as inputs to a neural network. Next, the neural network generates a dose prediction based on the at least one anatomical mask and the applicator mask. Then, the dose prediction is converted into one or more dwell positions and one or more corresponding dwell times for a radioactive source within an applicator to deliver a radiation treatment to a patient. Next, the radiation treatment is delivered, by a brachytherapy delivery unit, to the patient by causing the radioactive source to travel through the applicator to the one or more dwell positions while pausing at each dwell position for a corresponding dwell time. In another example, the neural network directly generates, based on the input masks, the one or more dwell positions and the one or more corresponding dwell times for the radioactive source within the applicator.

### CROSS-REFERENCE TO RELATED APPLICATIONS

This application is a national stage entry of Patent Cooperation Treaty Application No. PCT/US2023/080346 filed Nov. 17, 2023, entitled “AUTOMATED BRACHYTHERAPY TREATMENT PLANNING USING KNOWLEDGE-BASED DOSE ESTIMATIONS,” which claims priority to U.S. Provisional Application No. 63/384,302 filed Nov. 18, 2022, entitled “AUTOMATED BRACHYTHERAPY TREATMENT PLANNING”. The disclosures of which are incorporated herein by reference in their entirety.

### GOVERNMENT SUPPORT

This invention was made with government support under CA267068 awarded by the National Institutes of Health. The government has certain rights in the invention.

### TECHNICAL FIELD

The present disclosure generally relates to Brachytherapy treatment.

### BACKGROUND

In high dose-rate (HDR) Brachytherapy, a device known as an applicator is inserted through cavities in the body (such as through the vagina and cervix), which serves as a pathway for a radioactive source to travel directly into the tissue. Needles or catheters can also be inserted interstitially. Catheters may also be fixed to a device placed on the skin to produce a surface mould. The patient then receives some sort of imaging, such as a computed tomography (CT) or magnetic resonance imaging (MRI) scan, which allows clinicians to visualize the internal anatomy. Improvements in treatment are crucial because patients are typically waiting in discomfort and/or under sedation for their treatment.

### SUMMARY

In some implementations, at least one anatomical mask and an applicator mask are provided as inputs to a neural network. Next, the neural network generates a dose prediction based on the at least one anatomical mask and the applicator mask. Then, the dose prediction is converted into one or more dwell positions and one or more corresponding dwell times for a radioactive source within an applicator to deliver a radiation treatment to a patient. Next, the radiation treatment is delivered, by a brachytherapy delivery unit, to the patient by causing the radioactive source to travel through the applicator to the one or more dwell positions while pausing at each dwell position for a corresponding dwell time. In another example, the neural network directly generates, based on the at least one anatomical mask and the applicator mask, the one or more dwell positions and the one or more corresponding dwell times for the radioactive source within the applicator.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g., the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 illustrates a logical block diagram of a process for implementing an automated treatment plan, in accordance with some example implementations of the current subject matter;

FIG. 2 illustrates a block diagram of a system for training a neural network to generate a 3D dose prediction, in accordance with some example implementations of the current subject matter;

FIG. 3 illustrates a block diagram of a system for generating a 3D dose prediction and delivering a corresponding dose to a patient, in accordance with some example implementations of the current subject matter;

FIG. 4 illustrates the operation of an optimizer, in accordance with some example implementations of the current subject matter;

FIG. 5 illustrates the operation of an optimizer, in accordance with some example implementations of the current subject matter;

FIG. 6 illustrates equations for calculating the dose for a full applicator and the mean squared error of the difference between a computed 3D dose and a reference 3D dose over a plurality of voxels, in accordance with some example implementations of the current subject matter;

FIG. 7 illustrates an example of a process for operating an automated treatment delivery system, in accordance with some example implementations of the current subject matter;

FIG. 8 illustrates an example of a process for training a neural network to generate dose predictions, in accordance with some example implementations of the current subject matter;

FIG. 9 illustrates an example of a process for operating an optimizer, in accordance with some example implementations of the current subject matter;

FIG. 10 depicts an example of a system, in accordance with some example implementations of the current subject matter;

FIG. 11 depicts an example of a delineated tumor target and surrounding organs, in accordance with some example implementations of the current subject matter;

FIG. 12 depicts a comparison between an actual dose distribution and a predicted dose, in accordance with some example implementations of the current subject matter;

FIG. 13 depicts modeling accuracy quantified by averaging the difference between actual and predicted doses for voxels within clinically relevant dose bins, in accordance with some example implementations of the current subject matter;

FIG. 14 depicts a graph of dose prediction accuracy, in accordance with some example implementations of the current subject matter;

FIG. 15 depicts an example of an original clinical treatment plan, in accordance with some example implementations of the current subject matter.

FIG. 16 depicts an example of a treatment plan, including a set of dwell times and the 3D dose computed from those dwell times, produced with an optimizer, in accordance with some example implementations of the current subject matter;

FIG. 17 depicts dose distributions of an automated plan which closely match the clinically approved plan distribution, in accordance with some example implementations of the current subject matter; and

FIG. 18 illustrates an example of a process for operating an automated treatment delivery system, in accordance with some example implementations of the current subject matter.

### DETAILED DESCRIPTION

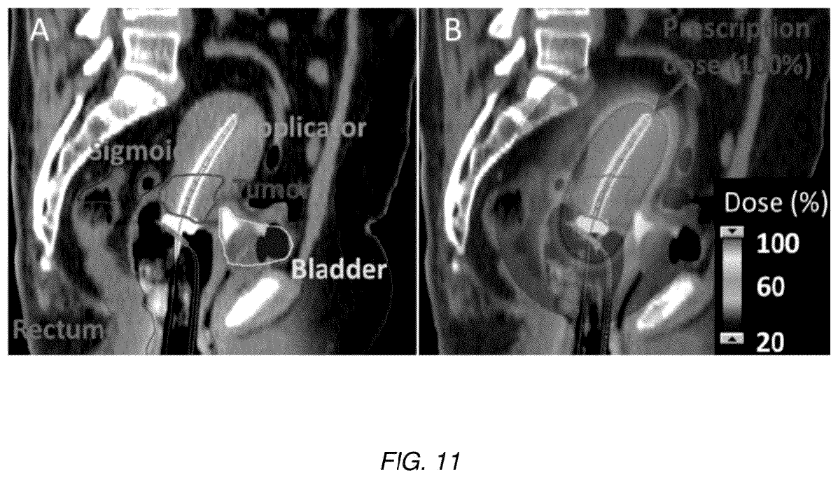

A patient undergoing Brachytherapy treatment typically receives some sort of imaging, such as a CT or MRI scan, which allows clinicians to visualize the internal anatomy. It is noted that the methods and systems presented herein do not rely on any particular imaging modality. After the imaging has been generated of the patient, clinicians will label tumor targets and important nearby healthy organs on the imaging (see left-side of FIG. 11). A treatment plan is then produced, in which the radiation is customized to the patient's anatomy using special software called a treatment planning system (see right-side of FIG. 11). The goal of this customization process is to maximize the radiation dose to regions suspected to contain cancer cells and minimize dose to the surrounding organs to prevent toxicity. During treatment delivery, a radioactive source will travel up the applicators, needles and/or catheters and pause in various locations (i.e., dwell positions) for varying amounts of time (i.e., dwell times). The longer the time spent in a location, the more radiation dose delivered to that region. Thus, a treatment plan consists of a recipe of dwell positions and dwell times that is transmitted to the brachytherapy equipment that controls the radioactive source.

Efficient treatment planning is crucial because patients are typically waiting in discomfort and/or under sedation for their treatment. There are currently very few tools that can adequately speed up the treatment planning process. Multiple methods for inverse optimization have been developed, in which an algorithm is used to determine optimal dwell times based on user-defined dose targets to the labelled tumor and anatomy.

Inverse optimization has been available for over a decade, yet it is not an automated solution for all types of brachytherapy because it has not consistently produced clinically acceptable treatment plans without significant manual refinement (a key failing of current methods). In cervical cancer, based on decades of clinical data, radiation is delivered to the delineated tumor target (shown in FIG. 11), but also the uterus and upper vagina and parametrium. As seen in FIG. 11, these other targets are not labeled on imaging, and the radiation dose not strictly conform to the tumor target. Thus, it is not straightforward for a non-human optimizer to determine a solution for dwell times that places dose in these other regions. Some groups have found work-arounds to preserve the desired dose shape in their inverse optimizations, such as delineating additional targets, while other solutions ignore this issue and produce non-standard dose distributions. Because of the challenges with inverse optimization, many centers instead perform brachytherapy treatment planning manually, which can take over an hour.

A few automated brachytherapy treatment planning solutions have been developed that make use of artificial intelligence (AI) concepts. Automated prostate brachytherapy treatment planning has been developed using anatomical feature metrics to identify the most similar patient from a training dataset. Optimized treatment plans were created using the training patient's dose as a starting point. While this solution may work well for low-dose rate prostate brachytherapy, it has not been applied to high-dose rate brachytherapy applications, where treatment planning is very different due to the ability to vary the individual dwell times of the source positions. Two other groups applied deep reinforcement learning to inverse optimization for cervical brachytherapy, but these methods likely suffer from the issues described above since they rely on delineated tumor targets.

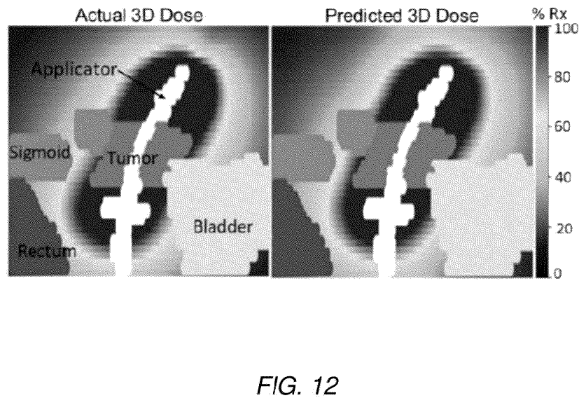

Knowledge-based planning is a concept that has been widely developed and implemented in the external-beam radiotherapy space, which relates treatment plan parameters to anatomical features. Models are trained on past patient data and applied to make predictions for new patients. Knowledge-based planning has been used clinically in external beam radiotherapy for plan quality control and automating treatment planning, and has been shown to significantly reduce treatment planning time, reduce plan quality variability, and improve plan quality. The application of knowledge-based planning to brachytherapy is more limited. Several articles have been published on simple one-dimensional (1D) models that predict the maximum dose to organs using anatomical geometry inputs, with application to cervical brachytherapy. A method has also been developed for three-dimensional (3D) dose prediction for one type of brachytherapy applicator (tandem-and-ovoids) using a convolutional neural network. FIG. 12 compares an actual dose distribution to the predicted dose using this model. Methods and mechanisms for converting these predictions to automated treatment plans are described in this disclosure. These methods and mechanisms include deriving dwell times from a known dose distribution, which is one element of automated brachytherapy using knowledge-based dose estimations.

Generally speaking, automated brachytherapy treatment planning using knowledge-based models (e.g., machine learning models) trained on past patient data may be implemented based on the methods and mechanisms presented herein. Automated brachytherapy treatment planning using knowledge-based models may take on multiple forms. In an example, 3D neural network dose predictions may be converted to dwell times. However, another example may involve directly predicting dwell times using a deep learning model, such as the methodology employed in human pose estimation or residual neural networks.

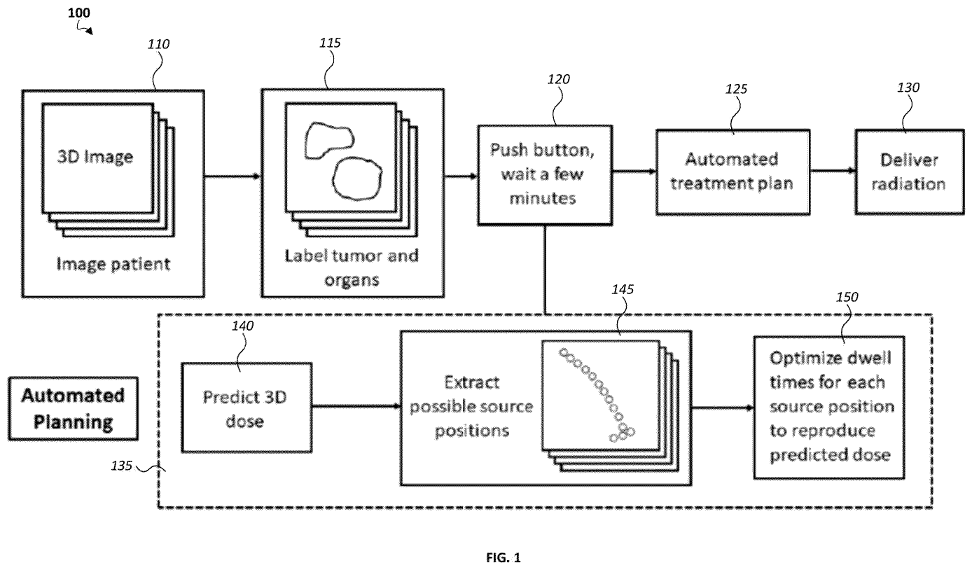

FIG. 1 depicts the overall automated planning using 3D dose predictions workflow. After imaging is captured, anatomy will be delineated on captured imaging. A button-click solution 120 will then produce an automated treatment plan 125 consisting of a series of dwell times for each dwell position within the applicator and/or needles. This treatment could then be immediately administered to a patient without any manual human adjustment.

In an example, automated planning includes the following components: (1) a mechanism to automatically pull anatomical image labels and applicator geometry from the treatment planning system, (2) automated generation of neural network inputs, (3) a neural network (or other type of machine learning model) that accurately predicts the 3D, patient-specific dose, (4) an optimizer that converts the 3D dose prediction into a set of dwell times, and (5) a mechanism to efficiently load optimized dwell times back into the treatment planning system for delivery. The specific methods to accomplish (1) and (5) will depend on the particular commercial treatment planning system. (2) represents a conversion of DICOM-RT to pre-processed data suitable for convolutional neural network training. (3) and (4) represent advances for completing the automated knowledge-based planning process and are described below.

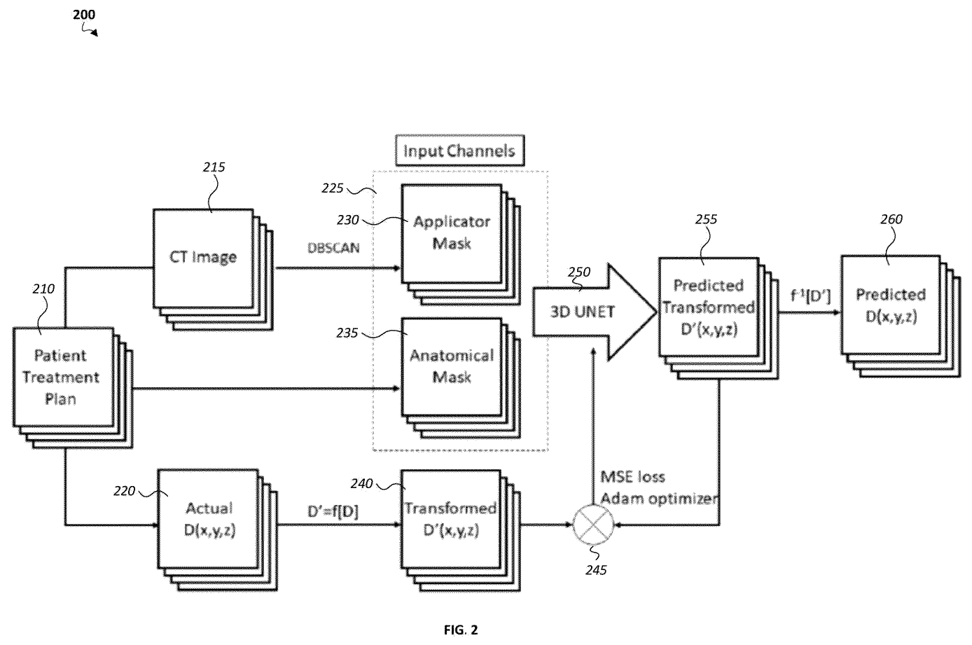

An example implementation of 3D dose prediction for tandem-and-ovoid cervical brachytherapy is shown in FIG. 2. The model employs a 3D U-Net architecture, which features both convolution and deconvolution layers. Two input channels are used-one indicating the 3D locations of tumor and organs (anatomical mask 235), and another locating the possible source positions (applicator mask 230). In an example, anatomical mask 235 is extracted from a treatment plan, and applicator mask 230 is generated using density-based spatial clustering of high intensity CT image voxels. Brachytherapy plans feature very large dose gradients near the applicator. In order to de-emphasize these high gradient regions with the mean squared error loss function, the dose input may be transformed using a function that scales down high doses.

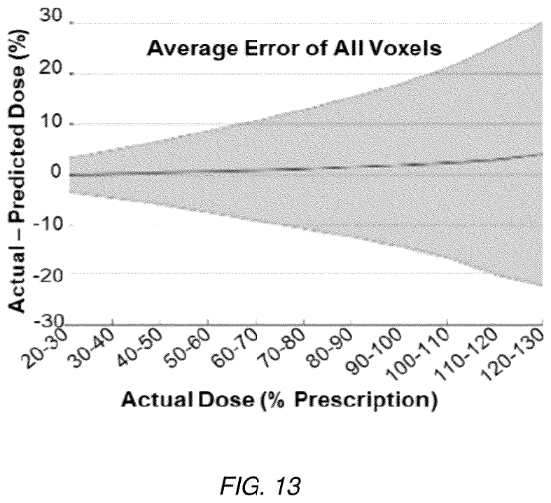

Modeling accuracy was quantified by averaging the difference between actual and predicted doses for voxels within clinically relevant dose bins (see FIG. 13). Mean absolute error over all voxels with doses from 0-150% of prescription was 9% for the independent test set. One type of model utilized only imaging data to derive application geometry, and additional inputs have been incorporated to improve predictive performance. For instance, a 3D dose kernel has been incorporated which depicts the shape of a dose distribution for a single dwell position. A set of additional 3D input matrices was produced by translating and rotating this kernel to the location of each dwell position. The benefits of using such an input are that dwell positions can be automatically pulled from a DICOM file, dwell positions may be easily translated to all types of brachytherapy applicators, needles and catheters, and dwell positions describe the physics of radiation delivery.

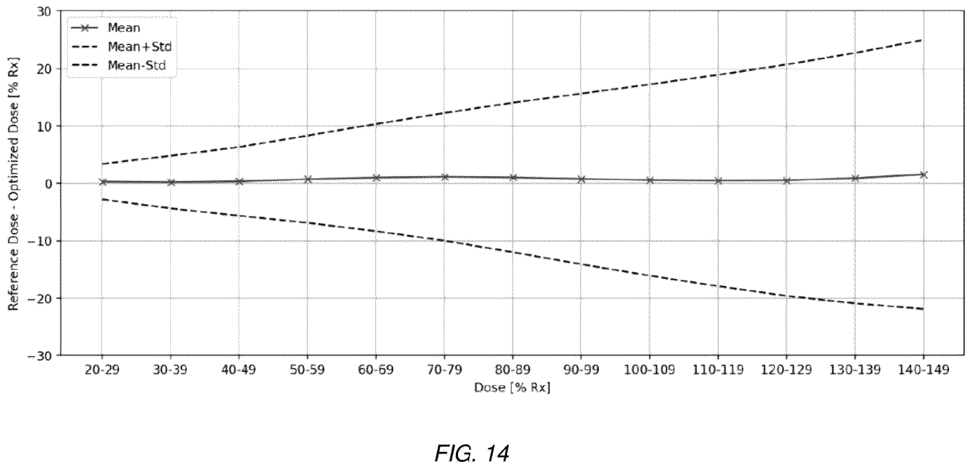

In an example, a Cascade U-Net neural network architecture may be implemented, which has been shown to provide superior dose prediction accuracy to standard 3D U-Nets for external beam radiotherapy. In an example, rather than utilizing a dose transformation, a mean squared error may be employed that down-weights high doses (for example, with a sigmoid function), data augmentation may be performed by flipping and translating subsets of the training data, and a cyclic learning rate may be used. The following changes have resulted in better dose prediction accuracy, as can be observed in the tighter error bars and reduced mean bias in FIG. 14.

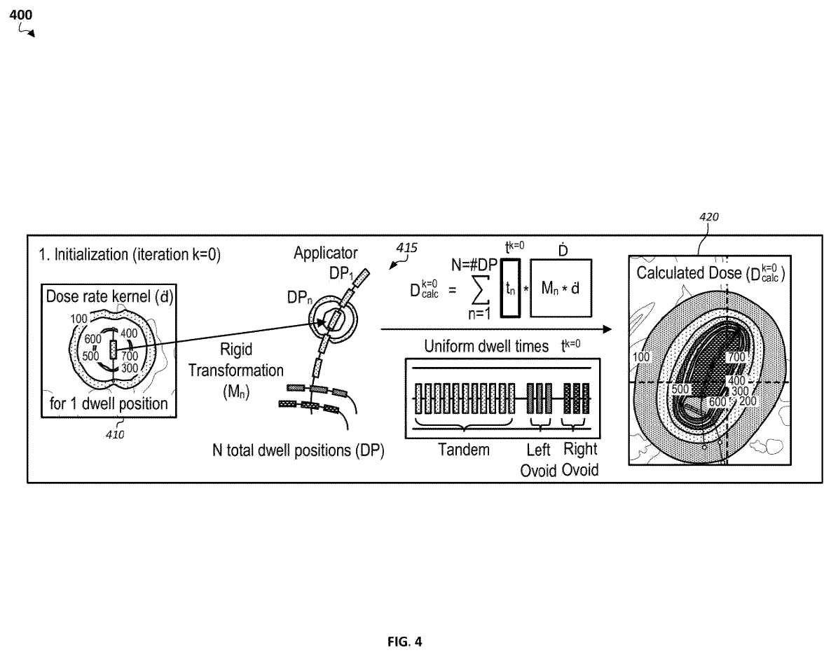

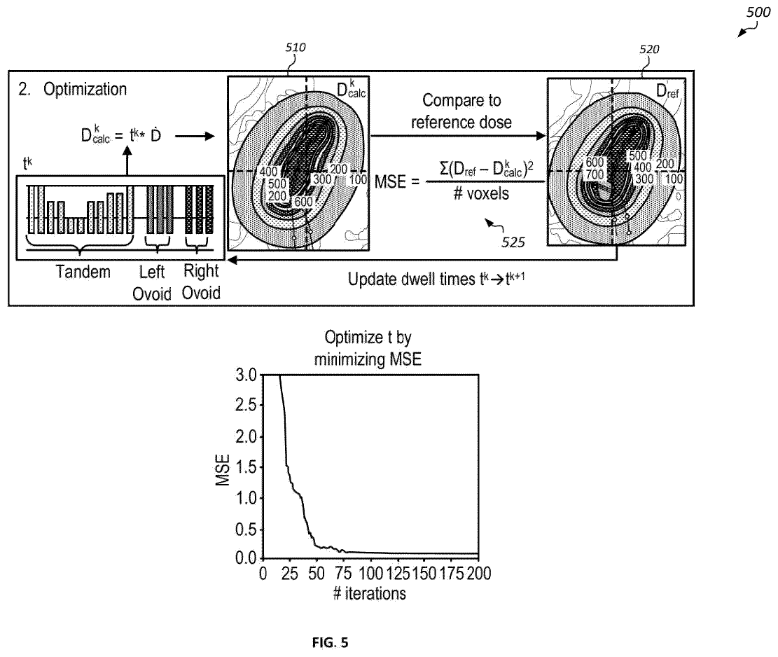

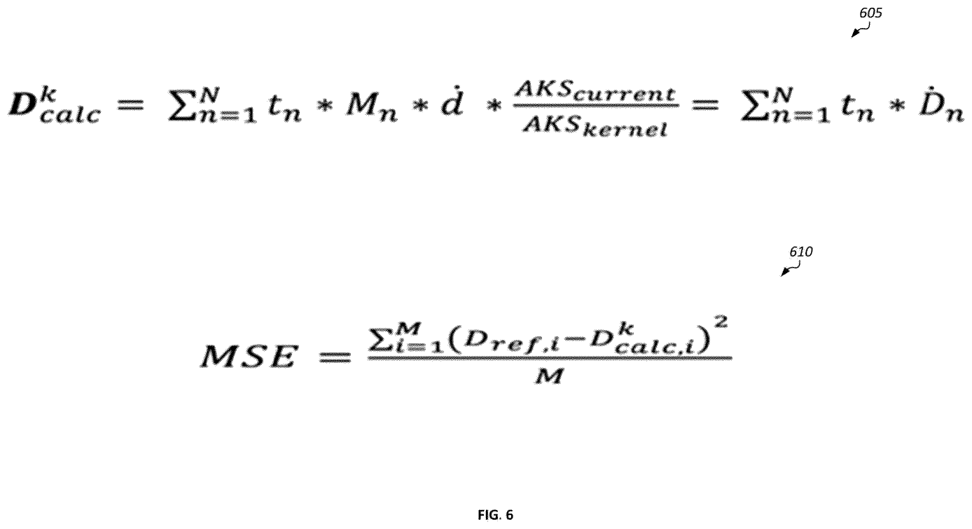

In an example, an optimizer iteratively determines the optimal dwell times required for a series of source positions to reproduce a given 3D dose distribution (see FIGS. 4-5). In order to compute a dose distribution prediction, a dose rate kernel 410 is produced by exporting the 3D dose for a single dwell position from the treatment planning system and normalizing by dwell time. In an example, the dose rate kernel 410 is a 400×400×400 matrix with 0.625×0.625×0.625 mm³ resolution. Dwell positions for a given patient's implant may be extracted from a plan DICOM file. By translating and rotating the dose rate kernel 410 to each dwell position in an applicator, needle or catheter, scaling by the dwell time for that position, summing over all dwell positions and scaling by the ratio of the air kerma strength at the time of the patient's treatment (AKS_{current}) to that of the kernel (AKSₖₑᵣₙₑₗ), the dose distribution for the full applicator is computed (D_{calc}). This is described in equation 605 of FIG. 6, where

$${D}_{calc}^{k}$$

is the 3D dose computed in the kth iteration, Mₙ is the matrix required to translate and rotate the dose rate kernel \dot{d} to the location of the nth dwell position and tₙ is the dwell time for that dwell position. These terms are summed over the N total dwell positions contained within the applicator (and needles or catheters, if present). Air kerma refers to the absorbed x-ray dose in air, and air kerma may be expressed in units of Gray.

To simplify computation, a dose rate matrix \dot{D}ₙ is computed once for each dwell position, which is then weighted by the updated dwell time with each iteration before summation. Dwell times are initialized with initial values and updated iteratively to minimize the mean squared error (MSE) between the calculated and reference doses as shown in equation 610 (of FIG. 6), where D_{ref,i} is the reference 3D dose for the ith voxel and M is the total number of voxels included in the computation. In an example, all voxels are included where the reference dose was between 80 to 120% of the prescription dose for that brachytherapy treatment fraction. In an example, a COBYLA (Constrained Optimization BY Linear Approximation) optimizer may be used, which is a numerical optimization method for constrained problems when the derivative of the objective function is not known.

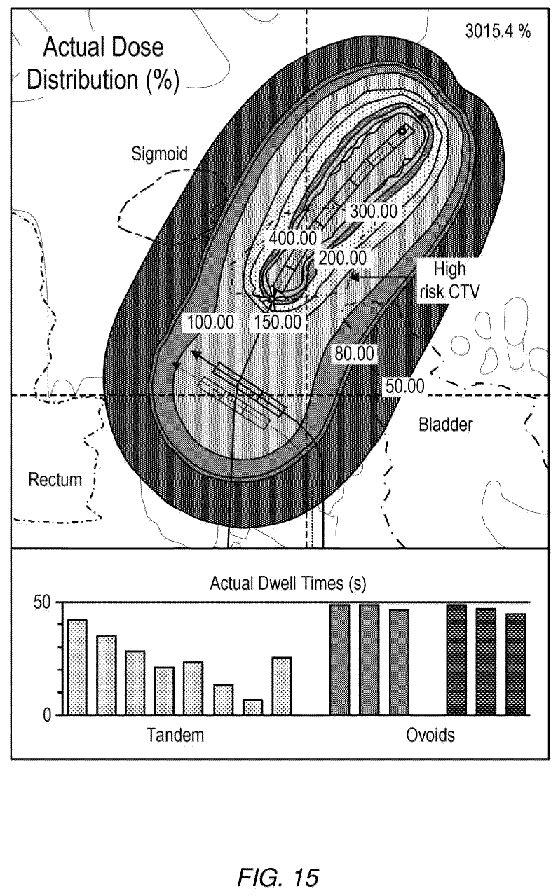

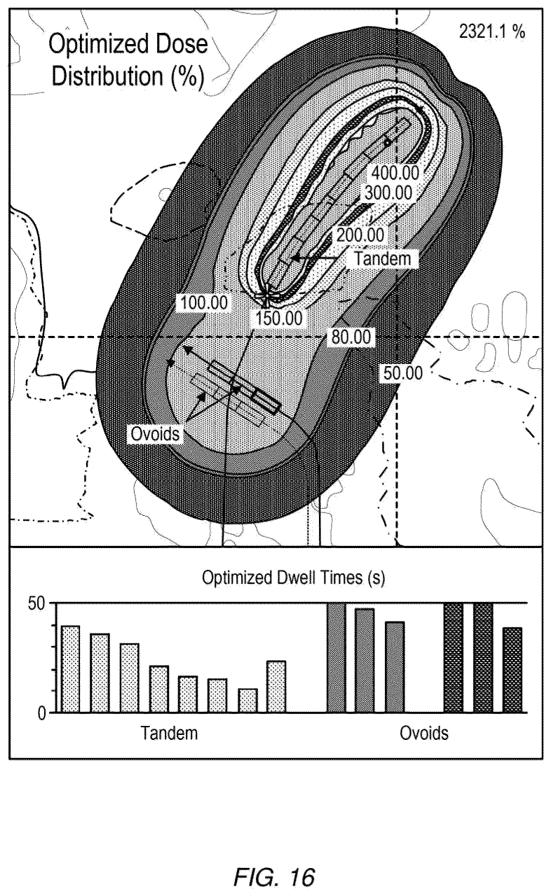

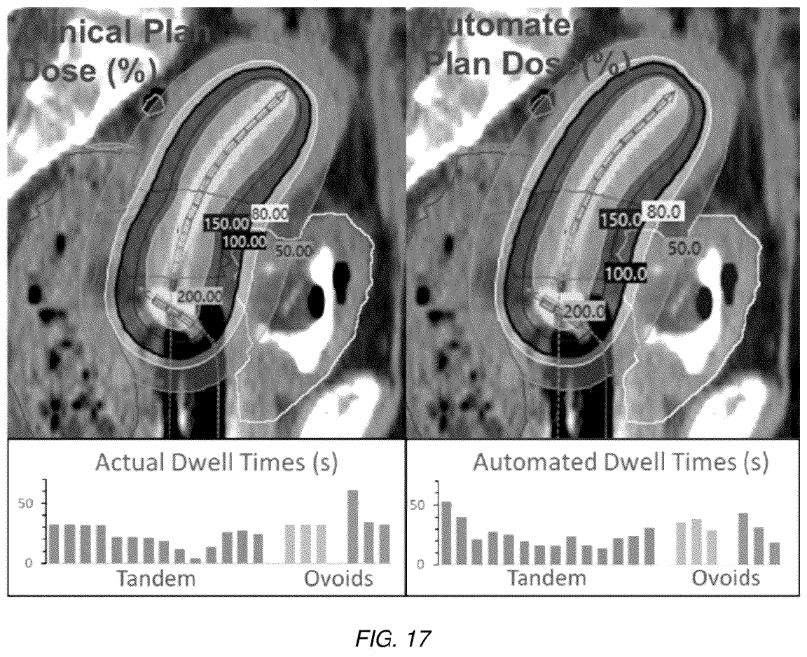

For each iteration, the actual optimization problem is approximated by linear programming problems, which are solved to obtain a possible solution. The solution is evaluated using the actual objective and constraint functions, yielding a new point in the optimization space. In an example, dwell times are constrained to fall between 0 and 100 s, since dwell times over 100 s are clinically uncommon. Multiprocessing may be used to speed up computation and individual dwell position dose rate matrices (\dot{D}ₙ), are generated in parallel. Full resolution \dot{D}ₙ are used to compute the final, optimized dose. Using a Windows computer with an Intel® Core™ i7-5930K CPU @ 3.50 GHz, 3501 Mhz, with 6 cores, 12 logical processors and 32 GB RAM, the optimization can typically be performed in 1 to 1.5 minutes. It has been demonstrated that the optimizer can reproduce clinical treatment plan dose and dwell times when the 3D dose from the clinical plan is fed in as the reference dose. An example of a treatment plan (i.e. a set of dwell times and the 3D dose computed from those dwell times) produced with the optimizer, compared to the original clinical treatment plan, is shown in FIGS. 15-16. It has also been demonstrated that automated treatment plans can be produced using the 3D dose predictions. The dose distributions of the automated plans closely match the clinically approved plan distribution and feature a reasonable set of dwell times (see FIG. 17). Some are clinically acceptable as-is, while others might require minimal manual adjustments to meet common clinical dose objectives. By employing the improved neural network, it is expected to be able to produce clinically acceptable treatment plans in under 5 minutes with a single button-click.

Automated planning has the potential to significantly reduce brachytherapy treatment planning time, which would decrease the time patients spend under anesthesia and reduce the usage of costly hospital resources, such as operating rooms and physician time. In addition to these benefits, automated planning can standardize the current human-driven treatment planning practice, reducing the variability in quality of treatments amongst patients and practitioners and potentially improving the quality of treatments overall. 3D dose prediction on its own could be used for plan quality control by providing optimal dose targets to aim for when treatment planning manually or using other methods. Models that are produced at high volume centers with brachytherapy expertise could be disseminated internationally to assist less experienced clinicians with producing more optimal, high quality treatment plans.

While the examples thus far have been described for high dose rate (HDR) brachytherapy, which uses an applicator or other device to temporarily guide a radioactive source into a patient, the current subject matter could also be applied to low dose rate (LDR) brachytherapy with radioactive seed implants. In this form of brachytherapy, the patient is imaged before an implant procedure is performed. A physician then labels the relevant anatomy on the imaging, and a treatment plan is developed with the goal of maximizing the radiation dose to the tumor target while minimizing the radiation dose to the surrounding anatomy. For HDR brachytherapy, the treatment parameters, determined during the planning process, consist of a set of dwell positions and dwell times. For LDR brachytherapy, the treatment parameters consist of the ideal locations for a series of radioactive seeds to be placed, as well as the ideal activity, or source strength, for each of these radioactive seeds. The seeds are then ordered and a physician will attempt to implant the seeds in the planned locations within the patient in an operating room.

The methods and mechanisms described herein may be adapted for application to LDR brachytherapy. In one example, using anatomical mask inputs, a neural network may be used to predict the ideal locations that radioactive seeds should be implanted in the body, as well as their corresponding activity. It is noted that the term “ideal location” may also be referred to herein as “preferred location”. It is also noted that the term “activity” may also be referred to herein as “source strength”.

In another example, a neural network may use the anatomical masks to predict the ideal or preferred dose distribution. An optimizer may then be used to determine the placement and activity of a series of radioactive seeds, in order to replicate the predicted dose distribution. A dose kernel may be used by the optimizer, but the optimizer would iteratively adjust the location of each dose kernel, as well as the intensity of the dose kernel, under a series of boundary conditions. For each iteration, a 3D dose would be calculated by summing over each dose kernel, weighted by its intensity scaling factor. 3D calculated dose would be compared to the 3D predicted dose using a metric such as mean squared error, and the optimizer would iteratively minimize this metric.

Referring now to FIG. 1, a block diagram of a process 100 for implementing an automated treatment plan is shown, in accordance with some example embodiments. In an example, at the beginning of process 100, in step 110, an imaging dataset of a patient is generated, using any suitable type of imaging mechanism. In an example, the imaging dataset includes one or more three-dimensional (3D) images of the patient. Next, the imaging dataset captured in step 110 is labeled in step 115, with the tumor and surround organs defined in the images. It is noted that the term “tumor” may also be used interchangeably herein with the term “target region”.

Then, in step 120, automatic generation of a treatment solution is initiated. In an example, the automatic generation of the treatment solution is initiated by pushing a button. In other examples, other mechanisms for initiating the automatic generation of the solution may be utilized, such as by sending a message, clicking a mouse while a cursor is in a particular location of a graphical user interface (GUI), tapping a particular location in a user interface of a mobile device, speaking a command to trigger a voice-activated mechanism, or other alternatives. The wait time for the solution to be generated may vary according to the embodiment.

Dashed box 135 illustrates one example of a sub-process that occurs in step 120 in response to the initiation of the automatic generation of the treatment solution. In an example, a 3D dose is predicted in step 140. This prediction may be generated by a machine learning engine, such as a neural network. The type of neural network may vary according to the embodiment. More details on the generation of the 3D dose prediction by the neural network will be provided later on in this disclosure. Next, in step 145, the possible source positions of an applicator, needle, or catheter for applying the dose to the patient are extracted from the treatment planning system. Then, in step 150, the dwell times are optimized for each source position to reproduce the predicted dose. In an example, the output of the automatic generation of the treatment solution shown in dashed box 135 is a set of dwell positions and corresponding dwell times. In other examples, the automatically generated treatment solution output may be any of various other types of machine parameters (e.g., a quantity of a treatment source to be delivered at each location of a plurality of locations).

After step 120, the set of dwell positions and corresponding dwell times are used to generate an automated treatment plan in step 125. The automated treatment plan is then used by a treatment delivery system to deliver a radiation treatment to the patient in step 130, by programming a radioactive source where to go within an applicator, needle or catheter and for how long.

Turning now to FIG. 2, a block diagram of a system 200 for training a neural network to generate a 3D dose prediction is shown, in accordance with some example embodiments. A patient treatment plan 210 is created for a specific patient. The patient treatment plan 210 includes some number of images 215 of the patient. The patient treatment plan 210 is used to create an anatomical mask 235, with anatomical mask 235 indicating 3D locations of a tumor, surrounding healthy tissue, one or more organs, and/or other anatomical regions of the patient. Also, an applicator mask 230 is generated, with applicator mask 230 specifying locations within the patient where an applicator, needle, or catheter is able to deliver a treatment source (e.g., radiation dose).

In an example, the applicator mask 230 and the anatomical mask 235 are the input channels 225 which are provided to a neural network 250. In an example, the neural network 250 is a 3D U-Net convolutional neural network. In other examples, other types of neural networks may be employed based on other types of architectures. In an example, the output of neural network 250 is a predicted transformed 3D dose 255. The predicted transformed 3D dose 255 is then transformed using a function that scales down high doses to create dose prediction 260. Once training of neural network 250 is complete, the dose prediction 260 generated by the trained neural network may be converted into a set of dwell positions and dwell times and then provided to a treatment delivery system to deliver the treatment source to the patient.

As used herein, the term “dose prediction” is defined as a prediction of a distribution of radiation that should be delivered over a particular 3D volumetric region of a patient to achieve a desired treatment outcome. The “dose prediction” may include a prediction of an amount of radiation to be delivered for each voxel within the 3D volumetric region specified for the patient. The granularity of the “dose prediction” may vary from implementation to implementation. It is noted that the terms “dose prediction” and “3D dose prediction” may be used interchangeably herein. Similarly, as used herein, the term “dose” is defined as an amount of radiation to be delivered to a patient, where the “dose” is calculated over a particular 3D volumetric region of the patient. The “dose” may include an amount of radiation to be delivered for each voxel within the 3D volumetric region specified for the patient.

Mean-square error (MSE) loss function 245 is shown at the bottom of FIG. 2 and is utilized during the training of the neural network 250. Actual 3D dose 220 is generated by a clinician based on the patient treatment plan 210. Actual 3D dose 220 is transformed into transformed 3D dose 240 which is compared to the predicted transformed 3D dose 255 generated by the neural network 250. The difference between transformed 3D dose 240 and predicted transformed 3D dose 255 is calculated over a plurality of voxels using a MSE loss function 245. The output of MSE loss function 245 is provided as feedback to neural network 250 to assist in the training of the neural network 250. During training, the weights of the various layers of the neural network 250 are adjusted so as to minimize the output of MSE loss function 245. In one example, an Adam optimizer is used to iteratively adjust these weights to minimize the loss function. The result of the training of the neural network 250 is the generation of predicted doses that more closely match the actual doses generated by a clinician. Neural network 250 may be trained by comparing actual 3D doses to corresponding predicted 3D doses for any number of patients.

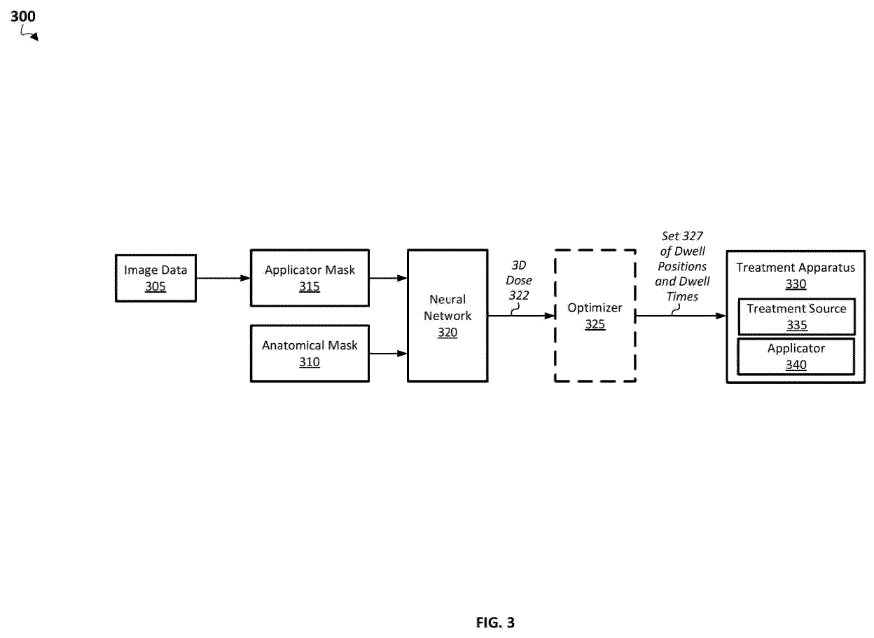

Referring now to FIG. 3, a block diagram of a system 300 for generating a 3D dose prediction and delivering a corresponding dose to a patient is shown, in accordance with some example embodiments. Image data 305 may be captured of a patient, with the image data captured using any of various suitable imaging mechanisms. In an example, image data 305 is then used to generate an applicator mask 315, with applicator mask 315 specifying locations within the patient where an applicator, needle, or catheter 340 is able to deliver the treatment source (e.g., radiation dose). Also, an anatomical mask 310 is generated, with anatomical mask 310 indicating 3D locations of a tumor and one or more organs of the patient.

In an example, applicator mask 310 and anatomical mask 315 are provided as inputs to a trained neural network 320. An example of training a neural network is shown in FIG. 2 and described in the corresponding description. Neural network 320 may have any suitable type of structure and/or architecture, with the structure and/or architecture varying from embodiment to embodiment. For example, neural network 320 may include any number and arrangement of convolution and deconvolution layers, with the numbers and arrangements of layers varying from embodiment to embodiment. Neural network 320 receives applicator mask 310 and anatomical mask 315 as inputs, and neural network 320 generates 3D dose 322 as an output based on the applicator mask 310 and anatomical mask 315 inputs.

In an example, 3D dose 322 may be optimized by an optional optimizer 325 that converts 3D dose 322 into a set 327 of dwell positions and dwell times. In another example, neural network 320 may be trained to generate the set 327 of dwell positions and dwell times based on the applicator mask 310 and anatomical mask 315 inputs. In either example, the set 327 of dwell positions and dwell times are provided as inputs to treatment apparatus 330 which delivers a treatment source 335 via applicator 340 to a patient based on the set 327 of dwell positions and dwell times. It is noted that treatment apparatus 330 may also be referred to herein as a brachytherapy delivery unit.

In an example, treatment apparatus 330 causes the treatment source 335 to move to each dwell position (in the set 327 of dwell positions and dwell times) within the applicator 340, which is located inside or on the patient. At each dwell position, treatment source 335 will pause for a corresponding dwell time while delivering the radiation to the patient. Once the corresponding dwell time has elapsed, the treatment apparatus 330 causes the treatment source 335 to move to the next dwell position (in the set 327 of dwell positions and dwell times) within the patient and pause for a corresponding dwell time while delivering the radiation to the patient. This process continues until each dwell position from the set 327 of dwell positions and dwell times for all applicators, needles or catheters has been reached and after treatment source 335 has remained at the dwell position for the specified dwell time. In an example, the treatment source 335 is a dose of radiation. In other examples, the treatment source 335 may be other types of doses, medicine, chemicals, or other material sources.

Turning now to FIG. 4, a diagram 400 illustrating the operation of an optimizer is shown, in accordance with some example embodiments. In an example, an optimizer (e.g., optimizer 325 of FIG. 3) iteratively determines the optimal dwell times required for a series of dwell positions to reproduce a given 3D dose distribution generated by a neural network (e.g., neural network 320 of FIG. 3). In an example, the optimizer may use a gradient descent algorithm to iteratively adjust the dwell times during the optimization process. In other examples, the optimizer may utilize other types of algorithms to adjust dwell times during the optimization process.

In an example, dose rate kernel 410 is produced by exporting the 3D dose for a single dwell position from a treatment planning system and normalizing by dwell time. In an example, dose rate kernel 410 is a 400×400×400 matrix with 0.625×0.625×0.625 mm³ resolution. In other examples, a dose rate kernel may be other sizes of matrices with other resolutions. Dwell positions for a given patient's implant may be extracted from a treatment plan DICOM file. Dwell position map 415 illustrates the N total dwell positions (DPs) for a given applicator where the given applicator is able to deliver the treatment source (e.g., radiation source).

By translating and rotating the dose rate kernel 410 to each dwell position in dwell position map 415 for an applicator, needle or catheter, scaling by the dwell time for that position, summing over all dwell positions in dwell position map 415 and scaling by the ratio of the air kerma strength at the time of the patient's treatment (ASK_{current}) to that of the kernel (ASKₖₑᵣₙₑₗ), the dose for the full applicator is computed (D_{calc}). The dose for the full applicator is shown as calculated dose 420. The calculations for determining calculated dose 420, in accordance with some embodiments, are shown in equation 605 of FIG. 6, where

$${D}_{calc}^{k}$$

is the 3D dose computed in the kth iteration, Mₙ is the matrix required to translate and rotate the dose rate kernel \dot{d} to the location of the nth dwell position, and tₙ is the dwell time for that dwell position. These terms are summed over the N total dwell positions of the applicator (and/or needles or catheters, if present).

Referring now to FIG. 5, a diagram 500 illustrating the operation of an optimizer is shown, in accordance with some example embodiments. Diagram 500 illustrates a later stage of the optimization process as compared to the initial stage shown in diagram 400 (of FIG. 4). As illustrated in diagram 500, the dwell times have been adjusted, over some number of iterations, to cause the computed 3D dose 510 (D_{calc}) to more closely match the reference 3D dose 520 (i.e., the 3D dose from the clinical plan). In an example, after multiple iterations with adjustments being made to the dwell times after each iteration, the final dwell times which are chosen are those dwell times which minimize the mean squared error (MSE) of the difference between the computed 3D dose 510 and the reference 3D dose 520 over the plurality of voxels. This is shown in equation 525 of FIG. 5 and in equation 610 of FIG. 6. The final dwell times which minimize the MSE may then be provided, along with the corresponding dwell positions, to a treatment delivery system to administer the treatment source to the patient. It is noted that the computed 3D dose 510 may also be referred to as a calculated 3D dose or a calculated 3D dose distribution. Similarly, the reference 3D dose 520 may also be referred to as a reference 3D dose distribution.

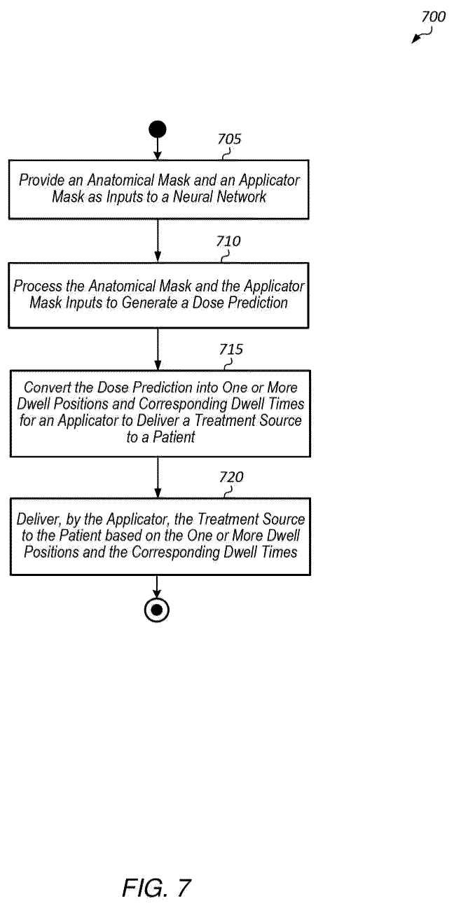

Referring now to FIG. 7, a process for operating an automated treatment delivery system is shown. At the start of method 700, an anatomical mask and an applicator mask are provided as inputs to a neural network (block 705). In an example, the anatomical mask indicates 3D locations of a tumor and one or more organs of a patient, and the applicator mask indicates regions within the patient where it is possible for an applicator to deliver a treatment source. In an example, the neural network is a U-Net neural network. In other examples, the neural network may be any of various other suitable types of neural networks (e.g., Cascade U-Net).

Next, the neural network processes the anatomical mask and the applicator mask inputs to generate a dose prediction (block 710). In an example, the dose prediction generated by the neural network is three-dimensional and patient-specific. Then, the dose prediction is converted into one or more dwell positions and corresponding dwell times for an applicator to deliver a treatment source to a patient (block 715). It is noted that the term “applicator” may refer to an applicator, needle, or catheter. As used herein, the term “applicator” may be defined as an instrument used to deliver brachytherapy. Different types of instruments which may be used to deliver brachytherapy include, but are not limited to, applicators, needles, and catheters. In an example, the treatment source is a radiation source. Next, the treatment source is delivered, by the applicator, to the patient based on the one or more dwell positions and the corresponding dwell times (block 720). In an example, at each dwell position within the applicator, the treatment source will pause for a corresponding dwell time while delivering the radiation to the patient. After the corresponding dwell time has elapsed, the treatment source will move to the next dwell position within the applicator. After block 720, method 700 ends.

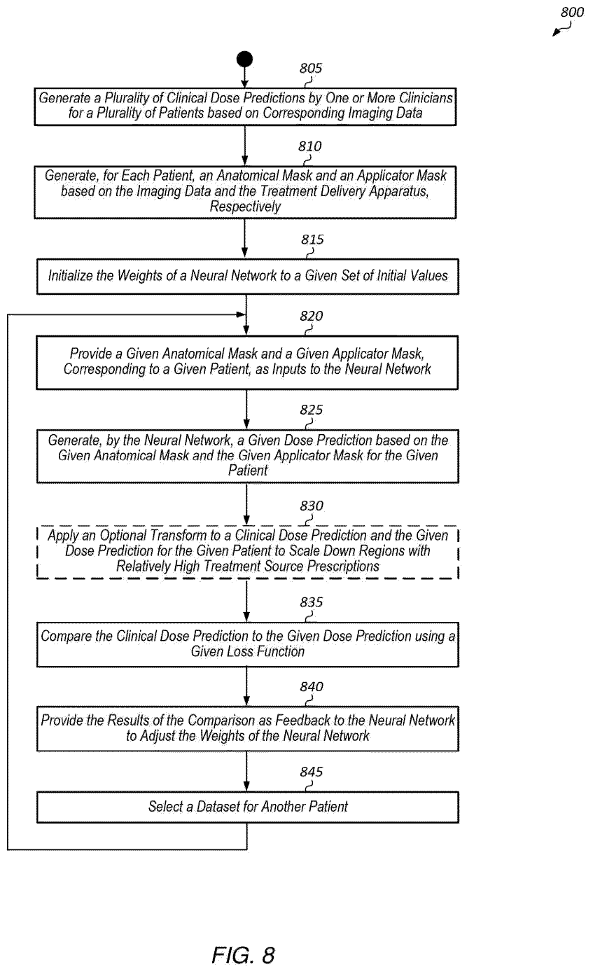

Turning now to FIG. 8, a process for training a neural network to generate dose predictions is shown. At the beginning of method 800, a plurality of clinical dose predictions are generated by one or more clinicians for a plurality of patients based on corresponding imaging data (block 805). Next, an anatomical mask and an applicator mask are generated for each patient based on the imaging data and the treatment delivery apparatus, respectively (block 810). Also, the weights (i.e., neurons) of a neural network are initialized to a given set of initial values (block 815). Starting with a first given patient, a given anatomical mask and a given applicator mask corresponding to the given patient are provided as inputs to the neural network (block 820). The neural network generates a given dose prediction based on the given anatomical mask and the given applicator mask for the given patient (block 825). An optional transform is applied to the clinical dose prediction and the given dose prediction for the given patient to scale down regions with relatively high treatment source prescriptions (block 830). Next, the clinical dose prediction is compared to the given dose prediction using a given loss function (block 835). In cases where the optional transform is applied to the clinical dose prediction and the given dose prediction to scale down relatively high doses, the transformed clinical dose prediction is compared to the transformed given dose prediction with the given loss function in block 835.

Then, the results of the comparison are provided as feedback to the neural network to adjust the weights of the neural network (block 840). Next, a dataset for another patient is selected (block 845), and then method 800 returns to block 820. Method 800 may continue for any number of iterations until the given dose predictions of the neural network converge on the clinical dose predictions, until the loss function is minimized over a given set of patient data, or until the difference between given dose predictions and clinical dose predictions is less than a threshold. In other examples, method 800 may continue until some other criteria is satisfied or until some other conditions are met.

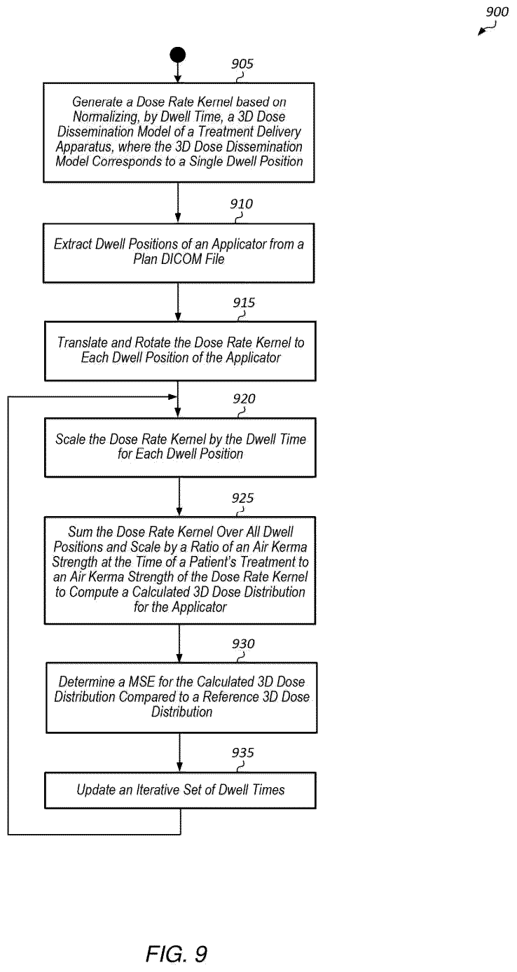

Referring now to FIG. 9, a process for operating an optimizer to iteratively determine optimal dwell times for a series of dwell positions to reproduce a given 3D dose distribution is shown. At the beginning of method 900, a dose rate kernel (e.g., dose rate kernel 410 of FIG. 4) is generated based on normalizing, by dwell time, a 3D dose dissemination model of a treatment delivery apparatus, where the 3D dose dissemination model corresponds to a single dwell position (block 905). In an example, the 3D dose dissemination model may be extracted from the treatment delivery apparatus. It is noted that the 3D dose dissemination model may vary from treatment delivery apparatus to treatment delivery apparatus. As used herein, the term “3D dose dissemination model” is defined as a model specifying how a treatment source (e.g., radiation) disseminates through the 3D space of a patient's anatomy for that particular treatment source and for a specific treatment delivery apparatus. It is noted that in another example, a lookup table may be used in place of the dose rate kernel, where the lookup table calculates the dose distribution to a point in space. In this example, a dose matrix may be calculated

Next, dwell positions of an applicator are extracted from a plan Digital Imaging and Communications in Medicine Standard (DICOM) file (block 910). Then, the dose rate kernel is translated and rotated to each dwell position of the applicator (block 915). Alternatively, a dose matrix may be calculated for each dwell position of the applicator, where the calculation of the dose matrix is based on a lookup table. Next, the dose rate kernel is scaled by the dwell time for each dwell position (block 920). Then, the dose rate kernel is summed over all dwell positions and scaled by a ratio of an air kerma strength at the time of a patient's treatment to an air kerma strength of the dose rate kernel to compute a calculated 3D dose distribution (e.g., computed 3D dose 510) for the applicator (block 925). Next, a mean squared error (MSE) is determined for the calculated 3D dose distribution compared to a reference 3D dose distribution (e.g., reference 3D dose 520) (block 930). Then, an iterative set of dwell times are updated (i.e., adjusted) (block 935), and then method 900 returns to block 920. Method 900 may continue with the iterative set of dwell times being updated for each iteration through method 900 until a set of dwell times is determined that minimizes the MSE between the calculated 3D dose distribution and the reference 3D dose distribution.

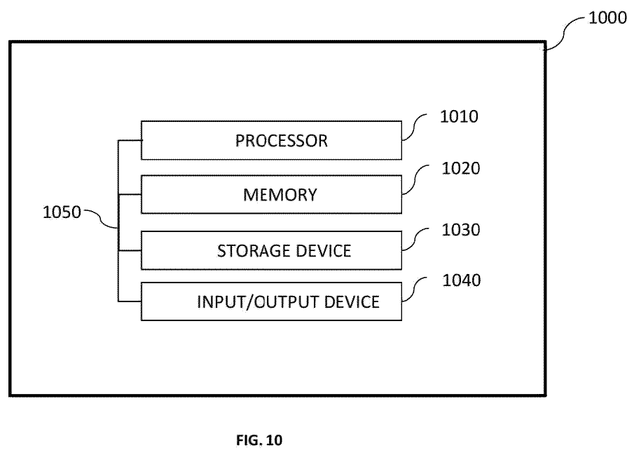

In some implementations, at least a portion of the current subject matter may be configured to be implemented in a system 1000, as shown in FIG. 10. The system 1000 may include a processor 1010, a memory 1020, a storage device 1030, and an input/output device 1040. Each of the components 1010, 1020, 1030 and 1040 may be interconnected using a system bus 1050. The processor 1010 may be configured to process instructions for execution within the system 1000. In some implementations, the processor 1010 may be a single-threaded processor. In alternate implementations, the processor 1010 may be a multi-threaded processor. The processor 1010 may be further configured to process instructions stored in the memory 1020 or on the storage device 1030, including receiving or sending information through the input/output device 1040. The memory 1020 may store information within the system 1000. In some implementations, the memory 1020 may be a computer-readable medium. In alternate implementations, the memory 1020 may be a volatile memory unit. In yet some implementations, the memory 1020 may be a non-volatile memory unit. The storage device 1030 may be capable of providing mass storage for the system 1000. In some implementations, the storage device 1030 may be a computer-readable medium. In alternate implementations, the storage device 1030 may be a floppy disk device, a hard disk device, an optical disk device, a tape device, non-volatile solid state memory, or any other type of storage device. The input/output device 1040 may be configured to provide input/output operations for the system 1000. In some implementations, the input/output device 1040 may include a keyboard and/or pointing device. In alternate implementations, the input/output device 1040 may include a display unit for displaying graphical user interfaces.

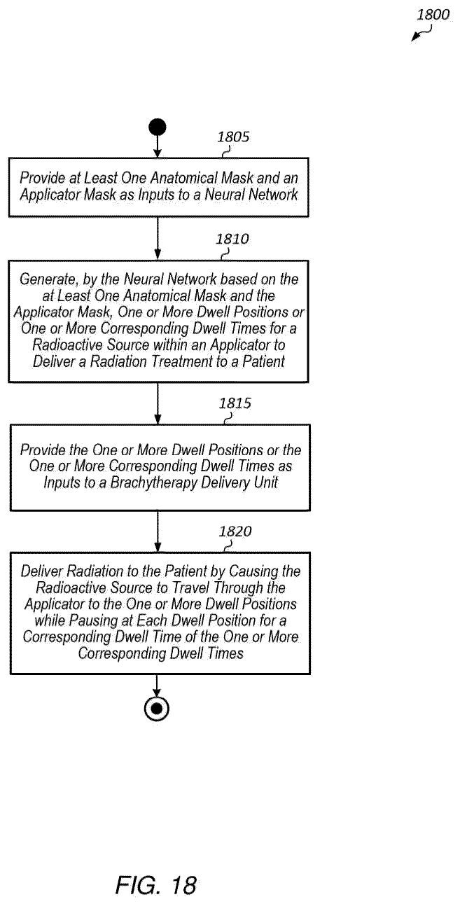

Turning now to FIG. 18, a process is shown for operating an automated treatment delivery system is shown. At the start of method 1800, at least one anatomical mask and an applicator mask are provided as inputs to a neural network (block 1805). In an example, each anatomical mask of the at least one anatomical mask indicates 3D locations of a tumor and one or more organs of a patient, and the applicator mask indicates regions within the patient where it is possible for an applicator to deliver a treatment source. In an example, the neural network is a U-Net neural network. In other examples, the neural network may be any of various other suitable types of neural networks (e.g., Cascade U-Net).

Next, the neural network generates, based on the at least one anatomical mask and the applicator mask, one or more dwell positions or one or more corresponding dwell times for a radioactive source within an applicator to deliver a radiation treatment to a patient (block 1810). In an example, the dose prediction generated by the neural network is three-dimensional and patient-specific. Then, the one or more dwell positions or the one or more corresponding dwell times are provided as inputs to a brachytherapy delivery unit (block 1815). It is noted that the term “brachytherapy delivery unit” may also be referred to as an applicator controller, radiation equipment, radiation controller, high-dose-rate (HDR) afterloader, or otherwise. Next, radiation is delivered to the patient by causing the radioactive source to travel through the applicator to the one or more dwell positions while pausing at each dwell position for a corresponding dwell time of the one or more corresponding dwell times (block 1820). In other words, radiation is emitted to the patient from the radioactive source within the applicator at each dwell position for the determined dwell time. After block 1820, method 1800 ends.

The systems and methods disclosed herein can be embodied in various forms including, for example, a data processor, such as a computer that also includes a database, digital electronic circuitry, firmware, software, or in combinations of them. Moreover, the above-noted features and other aspects and principles of the present disclosed implementations can be implemented in various environments. Such environments and related applications can be specially constructed for performing the various processes and operations according to the disclosed implementations or they can include a general-purpose computer or computing platform selectively activated or reconfigured by code to provide the necessary functionality. The processes disclosed herein are not inherently related to any particular computer, network, architecture, environment, or other apparatus, and can be implemented by a suitable combination of hardware, software, and/or firmware. For example, various general-purpose machines can be used with programs written in accordance with teachings of the disclosed implementations, or it can be more convenient to construct a specialized apparatus or system to perform the required methods and techniques.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

Although ordinal numbers such as first, second and the like can, in some situations, relate to an order; as used in a document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

The foregoing description is intended to illustrate but not to limit the scope of the invention, which is defined by the scope of the appended claims. Other implementations are within the scope of the following claims.

These computer programs, which can also be referred to programs, software, software applications, applications, components, or code, include program instructions (i.e., machine instructions) for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives program instructions as a machine-readable signal. The term “machine-readable signal” refers to any signal used to provide machine instructions and/or data to a programmable processor. The machine-readable medium can store such program instructions non-transitorily, such as for example as would a non-transient solid state memory or a magnetic hard drive or any equivalent storage medium. The machine-readable medium can alternatively or additionally store such machine instructions in a transient manner, such as would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user can provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including acoustic, speech, or tactile input.

The subject matter described herein can be implemented in a computing system that includes a back-end component, such as for example one or more data servers, or that includes a middleware component, such as for example one or more application servers, or that includes a front-end component, such as for example one or more client computers having a graphical user interface or a Web browser through which a user can interact with an implementation of the subject matter described herein, or any combination of such back-end, middleware, or front-end components. The components of the system can be interconnected by any form or medium of digital data communication, such as for example a communication network. Examples of communication networks include, but are not limited to, a local area network (“LAN”), a wide area network (“WAN”), and the Internet.

The computing system can include clients and servers. A client and server are generally, but not exclusively, remote from each other and typically interact through a communication network. The relationship of client and server arises by virtue of computer programs running on the respective computers and having a client-server relationship to each other.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

Example 1: A method, comprising: providing at least one anatomical mask and an applicator mask as inputs to a neural network; generating, by the neural network, a dose prediction based on the at least one anatomical mask and the applicator mask; converting the dose prediction into one or more dwell positions and one or more corresponding dwell times for a radioactive source within an applicator to deliver a radiation treatment to a patient; and delivering the radiation treatment to the patient based on the one or more dwell positions and the one or more corresponding dwell times.

Example 2: The method of Example 1, wherein the applicator mask specifies locations within a patient where it is possible for the radioactive source to be positioned within an applicator.

Example 3: The method of any of Examples 1, wherein the at least one anatomical mask indicates three-dimensional locations of a target region for the radiation treatment and one or more organs.

Example 4: The method of any of Examples 1-3, wherein delivering the radiation treatment to the patient comprises providing the one or more dwell positions and the one or more corresponding dwell times for a radioactive source to a brachytherapy delivery unit for administering the radiation treatment to the patient.

Example 5: The method of any of Examples 1-4, further comprising delivering the radiation treatment to the patient through the applicator while the radioactive source pauses at each dwell position of the one or more dwell positions for a corresponding dwell time.

Example 6: The method of any of Examples 1-5, wherein the dose prediction is intended to maximize the radiation dose to regions suspected to contain cancer cells while minimizing the radiation dose to surrounding organs to prevent toxicity.

Example 7: The method of any of Examples 1-6, wherein converting the dose prediction into the one or more dwell positions and the one or more corresponding dwell times comprises: translating and rotating a dose rate kernel to each dwell position of the one or more dwell positions; scaling the dose rate kernel by a corresponding dwell time for each dwell position of the one or more dwell positions; summing, based on the translating, rotating, and scaling, the dose rate kernel over the one or more dwell positions to determine a sum; scaling the sum by a ratio of a first air kerma strength at a time of treatment of the patient to a second air kerma strength of the dose rate kernel; determining a calculated dose distribution based on the scaling of the sum; adjusting an iterative set of dwell times over a plurality of iterations while determining a mean squared error between the calculated dose distribution and a reference dose distribution; and determining a final set of dwell times from a final calculated dose distribution which minimizes the mean squared error.

Example 8: The method of any of Examples 1-7, wherein the reference dose distribution is the dose prediction generated by the neural network.

Example 9: The method of any of Examples 1-8, further comprising generating the dose rate kernel based on normalizing, by dwell time, a three-dimensional dose dissemination model of a treatment delivery apparatus, where the three-dimensional dose dissemination model corresponds to a single dwell position.

Example 10: The method of any of Examples 1-9, wherein the dose prediction is three-dimensional and patient-specific.

Example 11: A system, comprising: at least one processor; and at least one memory including program instructions which when executed by the at least one processor causes operations comprising: providing at least one anatomical mask and an applicator mask as inputs to a neural network; generating, by the neural network based on the at least one anatomical mask and the applicator mask, one or more dwell positions or one or more corresponding dwell times for a radioactive source within an applicator to deliver a radiation treatment to a patient; providing the one or more dwell positions or the one or more corresponding dwell times as inputs to a brachytherapy delivery unit; and delivering, by the brachytherapy delivery unit, the radiation treatment to the patient by causing the radioactive source to travel through the applicator to the one or more dwell positions while pausing at each dwell position for a corresponding dwell time of the one or more corresponding dwell times.

Example 12: The system of Example 11, wherein the applicator mask specifies locations within a patient where it is possible for the radioactive source to be positioned within an applicator.

Example 13: The system of any of Examples 11-12, wherein the at least one anatomical mask indicates three-dimensional locations of a target region for the radiation treatment and one or more organs.

Example 14: The system of any of Examples 11-13, wherein the delivery of the radiation is intended to maximize the radiation treatment to regions suspected to contain cancer cells while minimizing the radiation treatment to surrounding organs to prevent toxicity.

Example 15: The system of any of Examples 11-14, wherein the applicator includes one or more of a catheter or a needle.

Example 16: The system of any of Examples 11-15, wherein the program instructions are further executable by the at least one processor to cause operations comprising generating, by the neural network based on the at least one anatomical mask and the applicator mask, the one or more dwell positions and the one or more corresponding dwell times for the radioactive source within the applicator to deliver the radiation treatment to the patient.

Example 17: The system of any of Examples 11-16, wherein the program instructions are further executable by the at least one processor to cause operations comprising providing the one or more dwell positions and the one or more corresponding dwell times as inputs to the brachytherapy delivery unit.

Example 18: The system of any of Examples 11-17, wherein the applicator is located inside the patient.

Example 19: The system of any of Examples 11-18, wherein the applicator is located on the patient.

Example 20: A non-transitory computer readable medium storing instructions, which when executed by at least one data processor, cause operations comprising: providing at least one anatomical mask and an applicator mask as inputs to a neural network; generating, by the neural network, a dose prediction based on the at least one anatomical mask and the applicator mask; converting the dose prediction into one or more dwell positions and one or more corresponding dwell times for a radioactive source within an applicator to deliver a radiation treatment to a patient; and delivering the radiation treatment to the patient based on the one or more dwell positions and the one or more corresponding dwell times.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations can be within the scope of the following claims.

What is claimed:

1. A method comprising:
providing at least one anatomical mask and an applicator mask as inputs to a neural network;
generating, by the neural network, a dose prediction based on the at least one anatomical mask and the applicator mask;
converting the dose prediction into one or more dwell positions and one or more corresponding dwell times for a radioactive source within an applicator to deliver a radiation treatment to a patient; and
delivering the radiation treatment to the patient based on the one or more dwell positions and the one or more corresponding dwell times.

2. The method of claim 1, wherein the applicator mask specifies locations within a patient where it is possible for the radioactive source to be positioned within an applicator.

3. The method of claim 1, wherein the at least one anatomical mask indicates three-dimensional locations of a target region for the radiation treatment and one or more organs.

4. The method of claim 1, wherein delivering the radiation treatment to the patient comprises providing the one or more dwell positions and the one or more corresponding dwell times for a radioactive source to a brachytherapy delivery unit for administering the radiation treatment to the patient.

5. The method of claim 1, further comprising delivering the radiation treatment to the patient through the applicator while the radioactive source pauses at each dwell position of the one or more dwell positions for a corresponding dwell time.

6. The method of claim 1, wherein the dose prediction is intended to maximize the radiation dose to regions suspected to contain cancer cells while minimizing the radiation dose to surrounding organs to prevent toxicity.

7. The method of claim 1, wherein converting the dose prediction into the one or more dwell positions and the one or more corresponding dwell times comprises:
translating and rotating a dose rate kernel to each dwell position of the one or more dwell positions;
scaling the dose rate kernel by a corresponding dwell time for each dwell position of the one or more dwell positions;
summing, based on the translating, rotating, and scaling, the dose rate kernel over the one or more dwell positions to determine a sum;
scaling the sum by a ratio of a first air kerma strength at a time of treatment of the patient to a second air kerma strength of the dose rate kernel;
determining a calculated dose distribution based on the scaling of the sum;
adjusting an iterative set of dwell times over a plurality of iterations while determining a mean squared error between the calculated dose distribution and a reference dose distribution; and
determining a final set of dwell times from a final calculated dose distribution which minimizes the mean squared error.

8. The method of claim 7, wherein the reference dose distribution is the dose prediction generated by the neural network.

9. The method of claim 7, further comprising generating the dose rate kernel based on normalizing, by dwell time, a three-dimensional dose dissemination model of a treatment delivery apparatus, where the three-dimensional dose dissemination model corresponds to a single dwell position.

10. The method of claim 1, wherein the dose prediction is three-dimensional and patient-specific.

11. A system, comprising:
at least one processor; and
at least one memory including program instructions which when executed by the at least one processor causes operations comprising:
providing at least one anatomical mask and an applicator mask as inputs to a neural network;
generating, by the neural network based on the at least one anatomical mask and the applicator mask, one or more dwell positions or one or more corresponding dwell times for a radioactive source within an applicator to deliver a radiation treatment to a patient;
providing the one or more dwell positions or the one or more corresponding dwell times as inputs to a brachytherapy delivery unit; and
delivering, by the brachytherapy delivery unit, the radiation treatment to the patient by causing the radioactive source to travel through the applicator to the one or more dwell positions while pausing at each dwell position for a corresponding dwell time of the one or more corresponding dwell times.

12. The system of claim 11, wherein the applicator mask specifies locations within a patient where it is possible for the radioactive source to be positioned within an applicator.

13. The system of claim 11, wherein the at least one anatomical mask indicates three-dimensional locations of a target region for the radiation treatment and one or more organs.

14. The system of claim 11, wherein the delivery of the radiation is intended to maximize the radiation treatment to regions suspected to contain cancer cells while minimizing the radiation treatment to surrounding organs to prevent toxicity.

15. The system of claim 11, wherein the applicator includes one or more of a catheter or a needle.

16. The system of claim 11, wherein the program instructions are further executable by the at least one processor to cause operations comprising generating, by the neural network based on the at least one anatomical mask and the applicator mask, the one or more dwell positions and the one or more corresponding dwell times for the radioactive source within the applicator to deliver the radiation treatment to the patient.

17. The system of claim 11, wherein the program instructions are further executable by the at least one processor to cause operations comprising providing the one or more dwell positions and the one or more corresponding dwell times as inputs to the brachytherapy delivery unit.

18. The system of claim 11, wherein the applicator is located inside the patient.

19. The system of claim 11, wherein the applicator is located on the patient.

20. A non-transitory computer readable medium storing instructions, which when executed by at least one data processor, cause operations comprising:
providing at least one anatomical mask and an applicator mask as inputs to a neural network;
generating, by the neural network, a dose prediction based on the at least one anatomical mask and the applicator mask;
converting the dose prediction into one or more dwell positions and one or more corresponding dwell times for a radioactive source within an applicator to deliver a radiation treatment to a patient; and
delivering the radiation treatment to the patient based on the one or more dwell positions and the one or more corresponding dwell times.
